(19) United States
(12) Patent Application Publication
(10) Pub. No.: US 2025/0346238 A1
(43) Pub. Date: Nov. 13, 2025

(54) MOTION SICKNESS PREVENTION DEVICE AND MOTION SICKNESS PREVENTION METHOD

(71) Applicant: TOYOTA BOSHOKU KABUSHIKI KAISHA, Aichi (JP)

(72) Inventors: Tomoya KITAZAKI, Aichi (JP); Shinji SUMITANI, Aichi (JP); Hiroyuki YAMASHIRO, Kyoto (JP)

(73) Assignee: TOYOTA BOSHOKU KABUSHIKI KAISHA, Aichi (JP)

(21) Appl. No.: 19/190,942

(22) Filed: Apr. 28, 2025

(30) Foreign Application Priority Data
May 10, 2024 (JP) 2024-077389

(51) Int. Cl. B60W 40/10; B60K 35/28

(52) U.S. Cl. CPC B60W 40/10; B60K 35/28; B60W 2540/221; B60W 2710/30

(57) ABSTRACT

Provided is a motion sickness prevention device capable of preventing a passenger from getting motion sickness. A motion sickness prevention device includes a sensor group, an obtaining section, a stimulus application device which applies a stimulus to a passenger of a moving body, and a control section which controls the stimulus application device. The control section controls the stimulus application device according to information which is related to behaviors of the moving body and which is obtained by the obtaining section, so as to apply a stimulus to the passenger.

This Nonprovisional application claims priority under 35 U.S.C. § 119 on Patent Application No. 2024-077389 filed in Japan on May 10, 2024, the entire contents of which are hereby incorporated by reference.

### TECHNICAL FIELD

The present disclosures relates to a motion sickness prevention device and a motion sickness prevention method.

### BACKGROUND ART

Conventionally, there has been a device which detects motion sickness of a passenger of a moving body such as a vehicle. For example, Patent Literature 1 discloses a motion sickness detection device which determines, on the basis of measurement of a temperature of a nose of a passenger, whether or not the passenger has motion sickness.

### CITATION LIST

### Patent Literature

### Patent Literature 1

Japanese Patent Application Publication, Tokukai, No. 2022-175384

### SUMMARY OF INVENTION

### Technical Problem

The motion sickness detection device disclosed in Patent Literature 1, however, cannot prevent the passenger from getting motion sickness, disadvantageously.

The present disclosure was made in view of the above problem, and has an objective to provide a motion sickness prevention device and a motion sickness prevention method each capable of preventing a passenger from getting motion sickness.

### Solution to Problem

In order to attain the above objective, a motion sickness prevention device in accordance with an aspect of the present disclosure includes: a sensor group which is provided to a moving body and which detects information related to behaviors of the moving body; an obtaining section which obtains the information related to the behaviors of the moving body detected by the sensor group; a stimulus application device which applies a stimulus to a passenger of the moving body; and a control section which controls the stimulus application device. The control section controls, according to the information related to the behaviors of the moving body obtained by the obtaining section, the stimulus application device so as to apply the stimulus to the passenger.

In order to attain the above objective, a motion sickness prevention method in accordance with an aspect of the present disclosure includes: a detection step of detecting, by a sensor group provided to a moving body, information related to behaviors of the moving body; an obtaining step of obtaining the information related to the behaviors of the moving body detected in the detection step; and a stimulus application step of applying a stimulus to a passenger of the moving body according to the information related to the behaviors of the moving body, the information being obtained in the obtaining step.

### Advantageous Effects of Invention

In accordance with an aspect of the present disclosure, it is possible to prevent a passenger from getting motion sickness.

### BRIEF DESCRIPTION OF DRAWINGS

FIG. 1 is a view schematically illustrating a configuration of a vehicle provided with a motion sickness prevention device in accordance with a first embodiment of the present disclosure.

FIG. 2 is a block diagram illustrating an example of a configuration of the motion sickness prevention device in accordance with the first embodiment.

FIG. 3 is a flowchart illustrating an example of a flow of a control process carried out by the motion sickness prevention device in accordance with the first embodiment.

FIG. 4 is a view illustrating an example of thresholds and index values with respective to respective behaviors of a vehicle in accordance with the first embodiment.

FIG. 5 is a view illustrating an example of correction values for thresholds with respect to respective pieces of biological information of a passenger in accordance with the first embodiment.

FIG. 6 is a view illustrating an example of correction values for thresholds with respect to respective pieces of vehicle information in accordance with the first embodiment.

FIG. 7 is a view illustrating an example of correction values for thresholds with respect to respective travel distances in accordance with the first embodiment.

FIG. 8 is a view illustrating an example of road conditions of a road on which a vehicle is scheduled to travel and index values thereof in accordance with the first embodiment.

FIG. 9 is a view illustrating an example of a relation between motion sickness characteristics of a passenger and correction values for thresholds in accordance with the first embodiment.

FIG. 10 is a flowchart illustrating an example of a flow of a motion sickness prevention process executed by the motion sickness prevention device in accordance with the first embodiment.

FIG. 11 is a view illustrating an example of a relation between content of reports given by a passenger and correction values for thresholds in accordance with the first embodiment.

FIG. 12 is a view illustrating a timing that a stimulus is applied by a motion sickness prevention device in accordance with the second embodiment.

### DESCRIPTION OF EMBODIMENTS

### First Embodiment

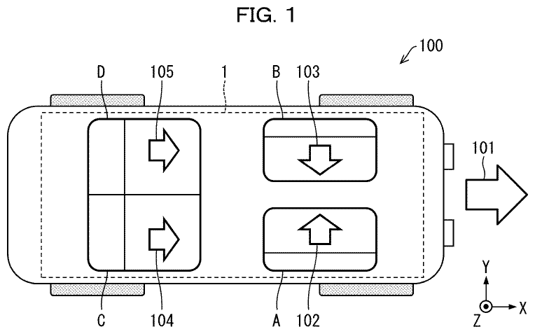

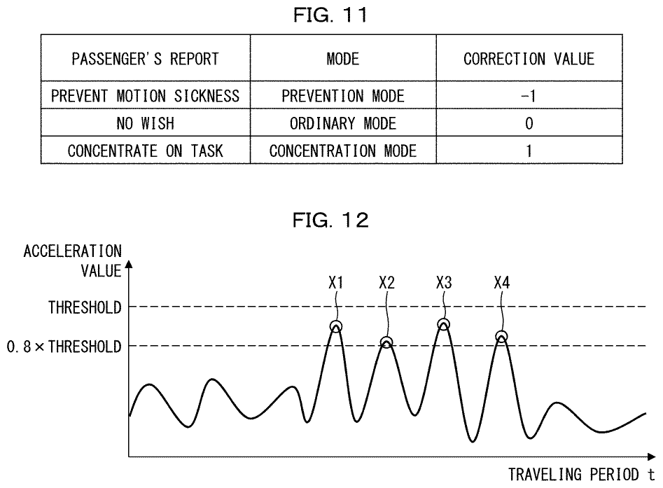

The following description will discuss, with reference to FIGS. 1 to 11, a motion sickness prevention device 1 in accordance with a first embodiment of the present disclosure.

### Motion Sickness Prevention Device

FIG. 1 is a view schematically illustrating a configuration of a vehicle 100 provided with the motion sickness prevention device 1. As shown in FIG. 1, the motion sickness prevention device 1 is provided to the vehicle 100. The vehicle 100 is one example of a moving body.

In the vehicle 100, a driver seat A, a front passenger seat B, a right rear seat C, and a left rear seat D are arranged. The motion sickness prevention device 1 is configured to be capable of individually applying a stimulus to passengers sitting on the driver seat A, the front passenger seat B, the right rear seat C, and the left rear seat D.

The vehicle 100 is a vehicle which can be provided with a full self-driving function. While the vehicle 100 is in a full self-driving mode, the passenger sitting on the driver seat A does not carry out any driving manipulation similarly to the passengers sitting on the front passenger seat B, the right rear seat C, and the left rear seat D. Meanwhile, when the vehicle 100 is switched to a mode which requires driving by passenger's manipulation and the vehicle 100 is no longer in the full self-driving mode, the passenger sitting on the driver seat A drives the vehicle 100.

Note that the present disclosure is not limited to the vehicle 100 having the full self-driving function, and is also applicable to vehicles in which various kinds of driving modes are selectable. Examples of the moving body include not only the vehicle 100 but also trains, ships, and air crafts.

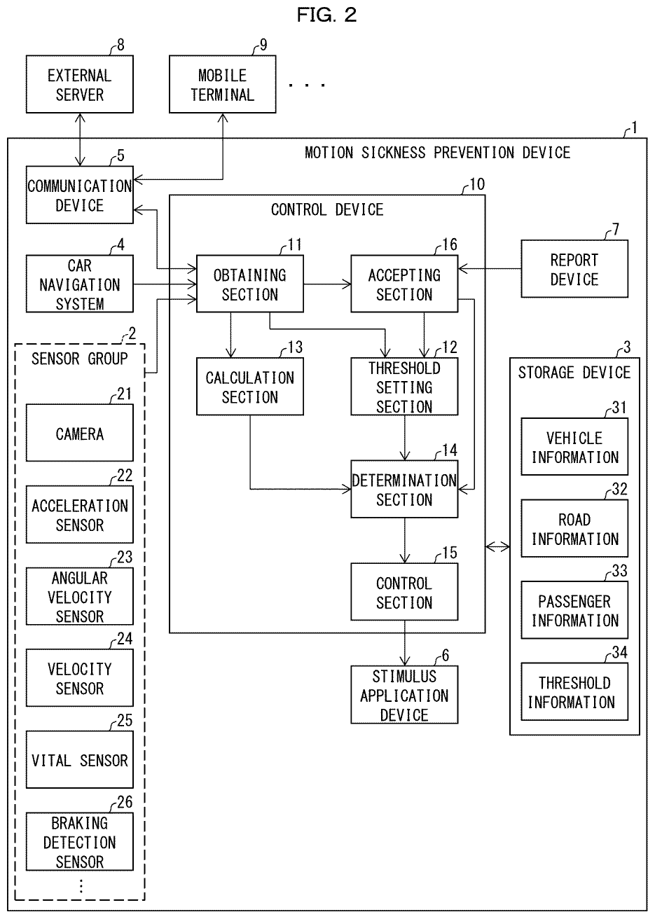

FIG. 2 is a block diagram illustrating an example of a configuration of the motion sickness prevention device 1. As shown in FIG. 2, the motion sickness prevention device 1 includes a sensor group 2, a storage device 3, a car navigation system 4, a communication device 5, stimulus application devices 6, report devices 7, and a control device 10. The control device 10 includes an obtaining section 11, a threshold setting section 12, a calculation section 13, a determination section 14, a control section 15, and an accepting section 16. The communication device 5 is communicably connected with an external server 8 and mobile terminals 9.

The motion sickness prevention device 1 obtains information related to behaviors of the vehicle 100 from the sensor group 2, the car navigation system 4, the external server 8, and the storage device 3 via the obtaining section 11. Further, the motion sickness prevention device 1 controls, by the control section 15, operation of the stimulus application device(s) 6 according to the information related to the behaviors of the vehicle 100, so as to apply a given stimulus to the passenger(s). With this, the motion sickness prevention device 1 prevents the passengers from getting motion sickness. It should be noted that the expression “prevent motion sickness” means not only to completely prevent motion sickness but also to reduce motion sickness even for just a little. This applies to similar expressions throughout the present specification.

### Sensor Group

The sensor group 2 includes a camera 21, an acceleration sensor 22, an angular velocity sensor 23, a velocity sensor 24, a vital sensor 25, and a braking detection sensor 26. The sensor group 2 transmits, to the obtaining section 11 of the control device 10, pieces of information detected by the sensors.

The camera 21 captures, for example, an image of an interior space of the vehicle 100 or an image of a space outside the vehicle 100. For example, the camera 21 detects pieces of face information of the passengers inside the vehicle 100 so as to authenticate the passengers. Further, by capturing an image of a space which is in front of the vehicle 100 and which is outside the vehicle 100, the camera 21 can detect a situation of traffic congestion on a road on which the vehicle 100 travels.

Furthermore, the camera 21 can be used as an orientation sensor which detects an orientation of a body of a passenger. The camera 21 is used to recognize a location of a passenger in the vehicle 100, particularly, an orientation of a body of the passenger. Note that the camera 21 may detect, on the basis of detection of orientations of the seats in the vehicle 100, orientations of bodies of the passengers sitting on the seats. Used as the orientation may be sensors which are integrated in the respective seats and which are each configured to detect sitting of a passenger.

The acceleration sensor 22 detects a degree of acceleration in a front-rear direction of the vehicle 100, a degree of acceleration in a left-right direction of the vehicle 100, and a degree of acceleration in an up-down direction of the vehicle 100. In the following description, as shown in FIG. 1, an axis extending along the front-rear direction of the vehicle 100 may sometimes be referred to as an “X-axis”, an axis extending along the left-right direction of the vehicle 100 may sometimes be referred to as a “Y-axis”, and an axis extending along the up-down direction of the vehicle 100 may sometimes be referred to as a “Z-axis”, for convenience of explanation.

The angular velocity sensor 23 detects a degree of an angular velocity around the X-axis, a degree of an angular velocity around the Y-axis, and a degree of an angular velocity around the Z-axis. The angular velocity sensor 23 is, for example, an oscillation-type gyro sensor.

Note that an angular velocity around the X-axis is referred to as a roll angular velocity, an angular velocity around the Y-axis is referred to as a pitch angular velocity, and an angular velocity around the Z-axis is referred to as a yaw angular velocity.

While the vehicle 100 is in a mode which requires driving by driver's manipulation, the braking detection sensor 26 detects braking manipulation carried out by the driver sitting on the driver seat A. Note that the sensor group 2 may include, in addition to the braking detection sensor 26, a steering wheel detection sensor which detects steering wheel manipulation carried out by the driver, an accelerator detection sensor which detects accelerator manipulation carried out by the driver, and/or the like.

### Storage Device

In the storage device 3, various kinds of information necessary for operation of the motion sickness prevention device 1 is stored. For example, in the storage device 3, vehicle information 31, road information 32, passenger information 33, threshold information 34, and/or the like are stored.

The vehicle information 31 includes, for example, information indicative of a type of the vehicle 100, a height of the vehicle 100, positions of the seats in the vehicle 100, vibration characteristics of the vehicle 100, a driving system of the vehicle 100, and/or the like. The road information 32 includes information indicative of a degree of undulation, a degree of a curve, the number of curves, and/or the like on the road on which the vehicle 100 travels.

The passenger information 33 includes, for example, pieces of face information, ID information, and motion sickness characteristics information of the passengers. Each of the pieces of the motion sickness characteristics information is information related to motion sickness of a passenger, and includes, for example, information related to a behavior of the vehicle 100 which is likely to cause motion sickness of the passenger, biological information of the passenger, and/or the like.

The threshold information 34 includes, for example, information indicative of thresholds having been set on the basis of the vehicle information 31, thresholds having been changed on the basis of the biological information of the passengers and the motion sickness characteristics of the passengers, thresholds having been changed on the basis of reports of the passengers, and/or the like. The thresholds are stored in the storage device 3 such that the thresholds are associated with the pieces of ID information of the passengers.

### Car Navigation System

The car navigation system 4 is a car navigation device mounted on the vehicle 100. The car navigation system 4 is one example of a navigation device which guides a passenger of the vehicle 100 along a route from a current location to a destination. The car navigation system 4 has, for example, map data stored therein. While the vehicle 100 is driving, the car navigation system 4 catches a current location by a Global Positioning System (GPS), displays the route on a screen, and provides the passenger with voice guidance for the route.

The car navigation system 4 transmits, to the obtaining section 11, the map data of a traveling route along which the vehicle 100 travels and the road information 32 indicative of a road condition(s) of the traveling route. The road information 32 transmitted from the car navigation system 4 to the obtaining section 11 is stored in the storage device 3. The road information 32 includes information indicative of a degree of undulation, a degree of a curve, the number of curves, and/or the like of the road on the traveling route. Note that the obtaining section 11 may obtain the road information 32 from the storage device 3.

### Communication Device

The communication device 5 is an interface via which the motion sickness prevention device 1 is connected with an external network such as the Internet. Via the communication device 5, the control device 10 of the motion sickness prevention device 1 and the external server 8 can be communicably connected with each other.

### Stimulus Application Device

Each of the stimulus application devices 6 is a device which applies a given stimulus to the passenger in the vehicle 100, so as to prevent the passenger from getting motion sickness. The stimulus application devices 6 are electrically connected with the control section 15. Operation of the stimulus application devices 6 is controlled by the control section 15. The stimulus application devices 6 are

respectively provided to the driver seat A, the front passenger seat B, the right rear seat C, and the left rear seat D of the vehicle 100. To be more specific, each of the stimulus application devices 6 is a device such as an outlet port of an air conditioner, a vibrator, a light, a speaker, or an aroma device provided to the seat. The stimulus application devices 6 individually apply, to the passengers, a stimulus such as an air blow, vibrations, light, sound, or aroma.

Note that each of the stimulus application devices 6 may be constituted by combination of some of the above-described devices to carry out output. Each of the stimulus application devices 6 may be a device other than the above. For example, each of the stimulus application devices 6 may be an air blowing device integrated into an adjusting mechanism of the respective seat of the vehicle 100 or the respective seat.

### Report Device

The report devices 7 are, for example, manipulation devices provided to the respective seats of the vehicle 100. By operating a corresponding one of the report devices 7, each passenger can select whether or not operation of a corresponding one of the stimulus application devices 6 is required. Further, each of the report devices 7 may be configured to allow adjustment of (i) an intensity of a stimulus that the stimulus application device 6 applies to the passenger and/or (ii) a period of application of the stimulus (hereinafter, the intensity and the period will be simply referred to as “an intensity and an application period of the stimulus application device 6”, respectively). Note that each of the report devices 7 may be a smartphone.

Further, each of the report devices 7 may be configured to allow selection of various operations modes of the stimulus application device 6. The operations modes of each of the stimulus application devices 6 include, for example, a “prevention mode” selected when the passenger does not want to get motion sickness, an “ordinary mode” selected when the passenger has no particular wish, and a “concentration mode” selected when the passenger wants to concentrate on a task. Examples of the task include manipulation of a smartphone, manipulation of a video game machine, and reading a book. Note that the kinds and number of the modes can be changed as appropriate.

Each of the report devices 7 may be a voice input device which accepts a voice from the passenger. By inputting a voice into a corresponding one of the report devices 7, each passenger can (i) select whether or not operation of a corresponding one of the stimulus application devices 6 is required and/or (ii) adjust the intensity and/or the application period of the corresponding one of the stimulus application devices 6. Each of the report devices 7 is not limited to the configuration that accepts an input individually given from the passenger. Alternatively, each of the report devices 7 may have a configuration that accepts wishes and/or the like of all the passengers input by manipulation carried out by a representative of all the passengers, for example, by the driver, the manipulation being carried out with respect to one of the report devices 7 which is provided to a display disposed near the driver seat.

### External Server

The external server 8 is electrically connected with the communication device 5 of the motion sickness prevention device 1. The external server 8 transmits, for example, biological information of a passenger and road information to the obtaining section 11 of the control device 10 via the communication device 5. (Mobile Terminal)

Each of the mobile terminals 9 is, for example, a smartphone, a tablet terminal, or a laptop personal computer. It is assumed that the mobile terminals 9 are carried by the respective passengers of the vehicle 100. The mobile terminals 9 can carry out radio communication with the communication device 5 to exchange various kinds of information therewith.

### Control Device

The control device 10 controls operation of the stimulus application devices 6 by obtaining, through the obtaining section 11, information from the sensor group 2, the car navigation system 4, and/or the like. The obtaining section 11 obtains information detected by the sensors in the sensor group 2. The obtaining section 11 obtains, for example, acceleration in the X-axis direction, acceleration in the Y-axis direction, acceleration in the Z-axis direction, a roll angular velocity, a pitch angular velocity, and a yaw angular velocity of the vehicle 100. Further, the obtaining section 11 obtains, from the sensor group 2, the car navigation system 4, and/or the like, road information 32 indicative of a road condition of the road on which the vehicle 100 travels. The obtaining section 11 obtains ID information of the passenger from the mobile terminal 9.

The threshold setting section 12 sets thresholds used for threshold determination carried out by the determination section 14. Specifically, the threshold setting section 12 sets thresholds for respective kinds of the behaviors of the vehicle 100. Examples of the kinds of the behaviors of the vehicle 100 include acceleration the X-axis direction, acceleration in the Y-axis direction, acceleration in the Z-axis direction, a roll angular velocity, a pitch angular velocity, and a yaw angular velocity. Further, on the basis of information such as the biological information of the passenger(s), the threshold setting section 12 changes the thresholds having been set.

The calculation section 13 calculates, for each passenger, an index value indicative of how likely the passenger is to get motion sickness. Specifically, the calculation section 13 calculates, on the basis of the information which is obtained by the obtaining section 11 and which is related to the behavior of the vehicle 100, index values for the passengers with respect to the respective kinds of the behaviors of the vehicle 100.

Each of the index values indicates an amount of accumulation of a factor which may cause motion sickness, and is indicated by a score, for example, “1”, “2”, or “3”. As the score increases, this means a stronger likelihood of getting motion sickness. Examples of a cause of motion sickness include repetition of rapid acceleration or rapid deceleration of the vehicle 100, rolling of the vehicle 100 in the front-rear direction and the left-right direction, and vibrations in the up-down direction. Other examples of the cause of motion sickness include factors related to biological information such as heart rates, respiration rates, and the like of the passengers.

For example, as shown in FIG. 1, in a case where the vehicle 100 is accelerated by self-driving in an X-axis positive direction as indicated by the arrow 101, the passenger's seat(s) may not face the front. In this case, the passengers sitting on the seats facing different directions have feelings of acceleration in different directions.

That is, the passenger sitting on the right rear seat C has a feeling of acceleration in the X-axis positive direction as indicated by the arrow 104, and the passenger sitting on the left rear seat D has a feeling of acceleration in the X-axis positive direction as indicated by the arrow 105. Meanwhile, the passenger sitting on the driver seat A has a feeling of acceleration in a Y-axis positive direction as indicated by the arrow 102, and the passenger sitting on the front passenger seat B has a feeling of acceleration in a Y-axis negative direction as indicated by the arrow 103.

In case where the directions of the acceleration felt by some of the passengers are different from the direction of acceleration of the vehicle 100 as in the above-described d situation, the calculation section 13 corrects, into the acceleration actually felt by the passengers, the acceleration applied to the vehicle 100, and then calculates index values on the basis of the corrected acceleration.

The determination section 14 determines whether or not each of the index values calculated by the calculation section 13 is equal to or more than a corresponding one of the thresholds set by the threshold setting section 12. In a case where any one of the index values is equal to or more than one of the thresholds corresponding to the one of the index values, the determination section 14 determines that it is in a state where the passenger is likely to get motion sickness.

In a case where the direction of the acceleration felt by each passenger is different from the direction of the acceleration of the vehicle 100, the determination section 14 determines whether or not each of the index values corrected by the calculation section 13 is equal to or more than a corresponding one of the thresholds which are in accordance with the orientation of the passenger's body.

The control section 15 controls operation of the stimulus application devices 6. In a case where the determination section 14 determines that any one of the index values is equal to or more than a threshold corresponding to the one of the index values, the control section 15 controls a corresponding one of the stimulus application devices 6 to apply a stimulus to the passenger.

The thresholds are respectively set for the kinds of the behaviors of the vehicle 100 which may cause motion sickness. For example, the thresholds are respectively set for acceleration in the X-axis direction, acceleration in the Y-axis direction, acceleration in the Z-axis direction, a roll angular velocity, a pitch angular velocity, and a yaw angular velocity.

The accepting section 16 accepts a passenger's report related to control of the stimulus application device 6. By manipulating a corresponding one of the report devices 7, each passenger transmits content of the report to the accepting section 16. Examples of the content of the report given by the passenger include a report stating that the passenger does not want to get motion sickness and a report stating that the passenger wants to concentrate on a task.

### Storage Device

In the storage device 3, the vehicle information 31, the road information 32, the passenger information 33, the threshold information 34, and/or the like are stored. The vehicle information 31 includes the information indicative of the type of the vehicle 100, the positions of the seats in the vehicle 100, the height of the vehicle 100, the vibration characteristics of the vehicle 100, the driving system of the vehicle 100, and/or the like. The road information 32 includes, for example, a travel distance from a departure point to a destination and/or a road condition(s) such as a curve(s) and a degree of undulation.

The passenger information 33 includes, for example, pieces of ID information allocated to the respective passengers. The threshold information 34 is information related to the thresholds for the passengers which thresholds have been changed along with traveling of the vehicle 100.

### Flow of Control Process

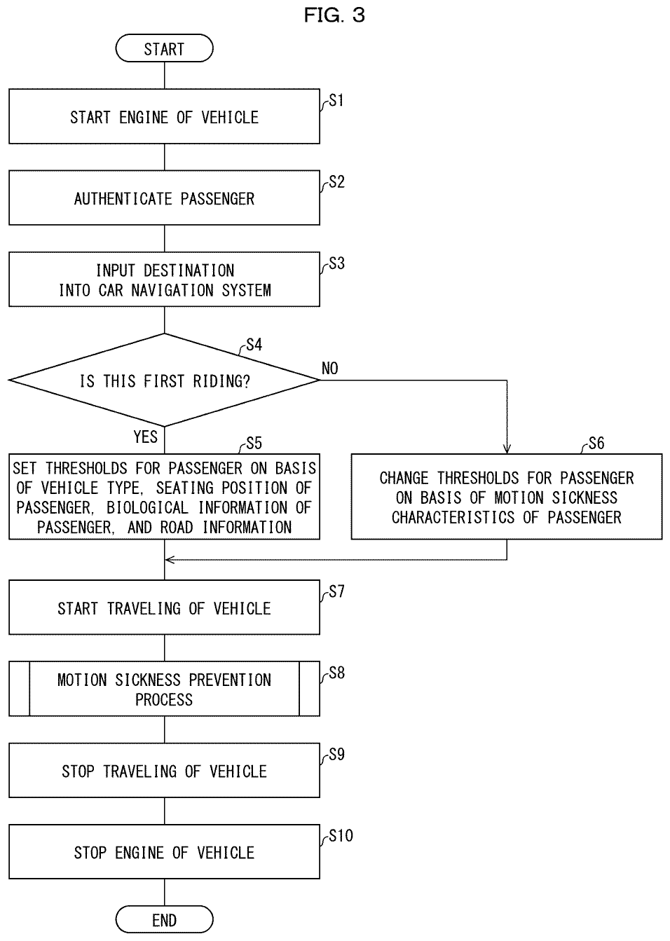

FIG. 3 is a flowchart illustrating an example of a flow of a control process carried out by the motion sickness prevention device 1. In the flowchart shown in FIG. 3, first, the passenger who is the driver starts an engine of the vehicle 100 (S1), and then the motion sickness prevention device 1 is activated. Alternatively, the motion sickness prevention device 1 may be activated by passenger manipulation.

After step S1, the motion sickness prevention device 1 captures, by, e.g. ., the camera 21, images of the faces of the passengers in the vehicle 100. Then, the control device 10 authenticates the passengers by comparing the pieces of face information obtained by the obtaining section 11 with the pieces of passenger information 33 in the storage device 3 (S2). The control device 10 may authenticate the passengers on the basis of pieces of information about the mobile terminals 9 carried by the passengers, the pieces of information being obtained by the obtaining section 11.

After step S2, in a case where a destination is input into the car navigation system 4 by passenger's manipulation (S3), the obtaining section 11 obtains, from the camera 21, the car navigation system 4, the external server 8, and the like, the road information 32 indicative of a road condition(s) in a traveling route along which the vehicle 100 travels, and stores the road information 32 in the storage device 3. The road information 32 includes, for example, information indicative of a travel distance, a road condition(s) such degrees and numbers of curves and undulations in the traveling route, and traffic congestion on the roads.

After step S3, the control device 10 determines whether or not the pieces of face information of the passengers authenticated in step S2 are included in the pieces of passenger information 33 stored in the storage device 3, so as to determine whether or not riding of each of the passengers is for the first time (S4). Note that the “riding for the first time” means a situation that threshold setting for a certain passenger by the motion sickness prevention device 1 is carried out for the first time since the certain passenger had never ridden the vehicle 100 provided with the motion sickness prevention device 1 and thus the threshold setting by the motion sickness prevention device 1 had not been carried out.

In a case where riding of a passenger is for the first time (S4: YES), the threshold setting section 12 sets thresholds for the passenger with respect to the respective behaviors of the vehicle 100 on the basis of the type of the vehicle 100 on which the passenger rides, a seating position of the passenger, the biological information of the passenger, and the road information 32 (S5). Then, the threshold setting section 12 stores, in the storage device 3, the thresholds thus set such that the thresholds are associated with ID information of the passenger. In a case where riding of the passenger is for the first time, the threshold setting section 12 may set thresholds for the passenger with respect to the respective behaviors of the vehicle 100 on the basis of a result of a questionnaire as to motion sickness which questionnaire is collected from the passenger in advance.

The information indicative of the type of the vehicle 100 is included in the vehicle information 31 stored in the storage device 3. The seating position of the passenger is detected by the camera 21. Note that the seating position of the passenger may be detected by known seating sensors provided to the respective seats. The biological information of the passenger is detected by the vital sensor 25. The road information 32 is stored in the storage device 3.

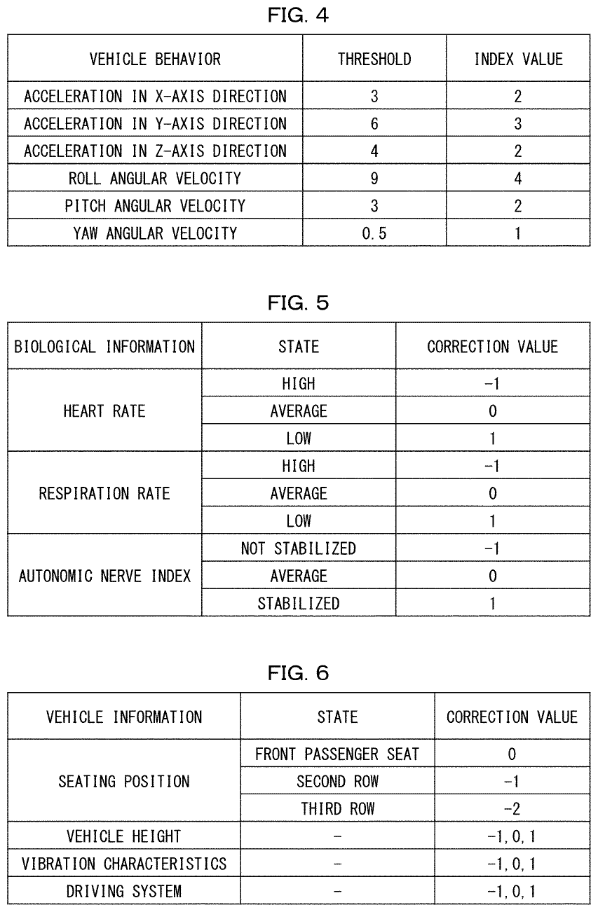

Note here that FIG. 4 is a view illustrating an example of thresholds and index values with respective to the respective behaviors of the vehicle 100. In the example shown in FIG. 4, the behaviors of the vehicle 100 include acceleration in the X-axis direction, acceleration in the Y-axis direction, acceleration in the Z-axis direction, a roll angular velocity, a pitch angular velocity, and a yaw angular velocity. Further, shown in FIG. 4 are initial values for the thresholds set with respect to the respective behaviors of the vehicle 100 and the index values with respect to the respective behaviors of the vehicle 100. In the example shown in FIG. 4, the index value of the yaw angular velocity is equal to or more than the threshold. Therefore, the stimulus application device 6 applies a stimulus to the passenger.

FIG. 5 is a view illustrating an example of correction values for the thresholds with respect to the respective pieces of biological information of a passenger. The biological information of the passenger includes a heart rate, a respiration rate, and an autonomic nerve index of the passenger. Each of the heart rate and the respiration rate is indicated by any of states, “high”, “average”, and “low”, each of which indicates a result of comparison with an average value of accumulated data. Each of the correction values for the thresholds refers to a score to be added to a threshold which has been already set.

For example, in a case where the heart rate and the respiration rate are high, considering that the passenger is likely to get motion sickness, “−1” is selected as the correction value for the thresholds, thereby facilitating the stimulus application device 6 to apply a stimulus. Meanwhile, in a case where the heart rate and the respiration rate are low, considering that the passenger is difficult to get motion sickness, “1” is selected as the correction value for the thresholds, thereby making it difficult for the stimulus application device 6 to apply a stimulus.

The autonomic nerve index is a value calculated on the basis of the heart rate and the respiration rate of a passenger. For example, in a case where the autonomic nerve index is not stabilized, considering that the passenger is likely to get motion sickness, “−1” is selected as the correction value for the thresholds, thereby facilitating the stimulus application device 6 to apply a stimulus. Meanwhile, in a case where the autonomic nerve index is stabilized, considering that the passenger is difficult to get motion sickness, “1” is selected as the correction value for the thresholds, thereby making it difficult for the stimulus application device 6 to apply a stimulus.

FIG. 6 is a view illustrating an example of correction values for thresholds with respect to the respective pieces of vehicle information 31. In the example shown in FIG. 6, indicated as the vehicle information 31 are the seating positions of the passengers, the height of the vehicle, the vibration characteristics, and the driving system. The threshold setting section 12 changes the thresholds by adding the correction values set with respect to the respective pieces of vehicle information 31 to the above-described initial values of the thresholds.

For example, in a case where a passenger sits on the right rear seat C or the left rear seat D, the correction value for the thresholds is “−1”. The intention in doing this is to facilitate the stimulus application device 6 to apply a stimulus by lowering the thresholds considering that a passenger sitting on a rear seat is generally more likely to get motion sickness than a passenger sitting on a front seat.

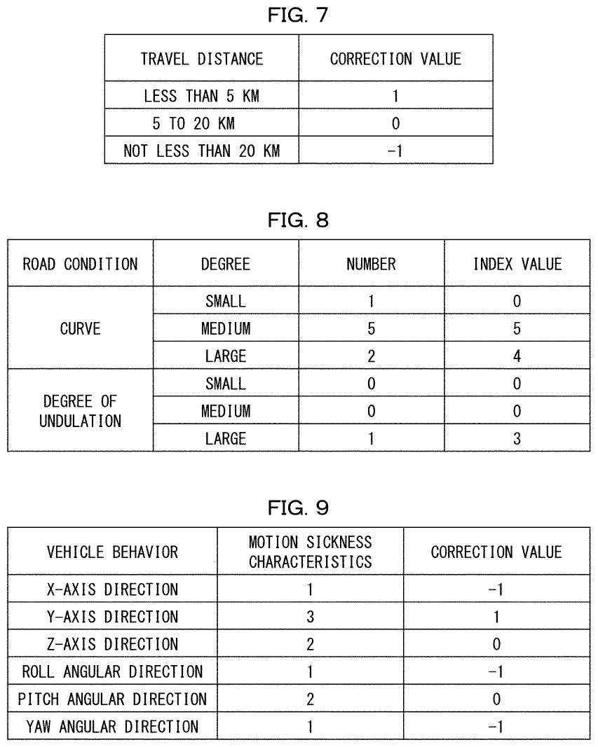

FIG. 7 is a view illustrating an example of correction values for thresholds with respect to respective travel distances. In the example shown in FIG. 7, as the travel distance increases, the correction value for the thresholds decreases. For example, in a case where the travel distance is less than 5 km, considering that the passenger is difficult to get motion sickness, the correction value for the thresholds is “1”, thereby making it difficult for the stimulus application device 6 to apply a stimulus. Meanwhile, in a case where the travel distance is not less than 20 km, considering that the passenger is likely to get motion sickness in this situation, the correction value for the thresholds is “−1”, thereby facilitating the stimulus application device 6 to apply a stimulus.

In a case where the road information 32 includes information indicating that traffic is congested on a road on which the vehicle is scheduled to travel, it is assumed that acceleration or deceleration of the vehicle 100 frequently occurs. Thus, the threshold setting section 12 lowers the threshold related to the acceleration in the X-axis direction.

FIG. 8 is a view illustrating an example of road conditions of the road on which the vehicle is scheduled to travel and index values thereof. When a passenger inputs a departure point and a destination into the car navigation system 4, a traveling route of the vehicle 100 is determined. In the traveling route shown in FIG. 8, it is shown that the index value increases in a part including more curves and the index value increases in a part having a higher degree of undulation.

In the storage device 3, information related to a road condition of a road in a traveling route along which a passenger traveled in the past and information related to motion sickness are stored. The threshold setting section 12 refers to a past traveling route having a road condition similar to that of a traveling route to be traveled this time, and determines a correction value for the thresholds.

For example, in a case where the traveling route to be traveled this time is similar to a traveling route in which the passenger got motion sickness in the past, the threshold setting section 12 selects “−1” as the correction value for the thresholds, thereby facilitating the stimulus application device 6 to apply a stimulus. Meanwhile, in a case where the traveling route to be traveled this time is similar to a traveling route in which the passenger did not get motion sickness in the past, the threshold setting section 12 selects “1” as the correction value for the thresholds, thereby making it difficult for the stimulus application device 6 to apply a stimulus.

Then, referring back to the flowchart in FIG. 3, if riding of the passenger on the vehicle is not for the first time (S4: NO), the threshold setting section 12 changes the thresholds for the passenger on the basis of the motion sickness characteristics of the passenger (S6). If riding of each passenger on the vehicle is not for the first time, the threshold setting section 12 changes the thresholds for the each passenger, the thresholds being determined in consideration of the motion sickness characteristics of the passenger and being stored in the storage device 3.

FIG. 9 is a view illustrating an example of a relation between motion sickness characteristics of a passenger and correction values for thresholds. In the example shown in FIG. 9, the behaviors of the vehicle are the X-axis direction, the Y-axis direction, the Z- axis direction, a roll angular direction, a pitch angular direction, and a yaw angular direction. Further, the motion sickness characteristics of the passenger with respect to acceleration in the respective directions are indicated by numerical values. As the numerical value increases, this means a higher difficulty of getting motion sickness.

For example, in a case where the motion sickness characteristics in the Y-axis direction is “3”, considering that the passenger is difficult to get motion sickness in response to the acceleration in the Y-axis direction, the correction value for the thresholds is “1”, thereby making it difficult for the stimulus application device 6 to apply a stimulus. Meanwhile, in a case where the motion sickness characteristics in the yaw angular direction is “1”, considering that the passenger is likely to get motion sickness in response to the acceleration in the yaw angular direction, the correction value for the thresholds is “−1”, thereby facilitating the stimulus application device 6 to apply a stimulus.

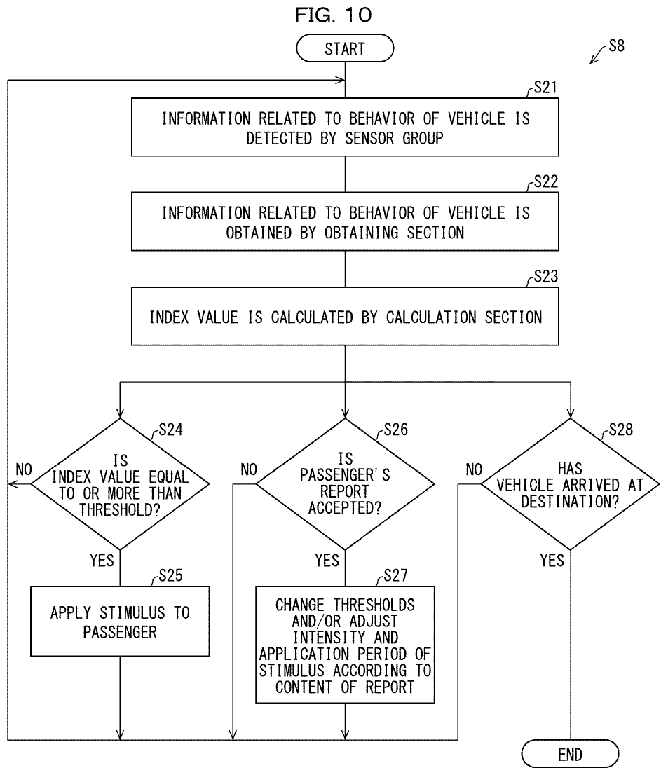

Then, referring back to the flowchart in FIG. 3, after step S5 or step S6, traveling of the vehicle 100 is started by driving manipulation carried out by the driver (S7). After step S7, while the vehicle 100 is traveling, a motion sickness prevention process (S8) shown in FIG. 10 is continuously executed by the motion sickness prevention device 1.

### Motion Sickness Prevention Process

The following description will discuss, with reference to FIG. 10, details of the motion sickness prevention process in step S8 shown in FIG. 3. FIG. 10 is a flowchart illustrating an example of a flow of the motion sickness prevention process (S8) executed by the motion sickness prevention device 1.

An objective of the motion sickness prevention process (S8) is to apply a stimulus to passengers according to a behavior of the vehicle 100 before the passengers get motion sickness, thereby preventing or reducing accumulation of a factor which may cause motion sickness of the passengers.

In the flowchart shown in FIG. 10, the sensor group 2 detects information related to behaviors of the vehicle 100 (S21: detection step). In step S21, for example, the acceleration sensor 22 detects acceleration in the front-rear direction, the left-right direction, and the up-down direction of the vehicle 100. The angular velocity sensor 23 detects a roll angular velocity, a pitch angular velocity, and a yaw angular velocity. The velocity sensor 24 detects a velocity in the front-rear direction of the vehicle 100. The vital sensor 25 detects a heart rate, a respiration rate, and/or the like of a passenger.

After step S21, the obtaining section 11 obtains information related to the behaviors of the vehicle 100 detected by the sensor group 2 in step S21 (S22: obtaining section). In step S22, the obtaining section 11 obtains (i) the acceleration in the front-rear direction, the left-right direction, and the up-down direction of the vehicle 100, (ii) the roll angular velocity, the pitch angular velocity, and the yaw angular velocity, and (iii) the heart rate, the respiration rate, and/or the like of the passenger.

After step S22, on the basis of the information related to the behaviors of the vehicle 100 obtained by the obtaining section 11 in step S22, the calculation section 13 calculates index values each indicative of a likelihood of getting motion sickness (S23). After step S23, steps S24, S26, and S28 are executed in parallel.

In step S24, the determination section 14 determines whether the index values calculated in step S23 are equal to or more than the respective thresholds set in step S4 or step S5.

In a case where the determination section 14 determines that at least one of the index values is equal to or more than a corresponding one of the thresholds (S24: YES), the control section 15 controls the stimulus application device 6 to apply a given stimulus to the passenger for a short time (S25: stimulus application step). Examples of the given stimulus include an air blow from the air conditioner, a sound from the speaker, vibrations from the vibrating device, and light emission from the light. The “short time” is, for example, approximately 10 seconds. Note that the period in which a stimulus is applied is adjustable by the passenger.

If the determination section 14 determines that the index values are less than the thresholds (S24: NO) or after step S25, steps S21 to S23 are repeated at given time intervals.

Subsequently, in step S26, the determination section 14 determines whether or not a report given by the passenger is accepted by the accepting section 16. The passenger manipulates the report device 7 to give a report.

If the report given by the passenger is accepted by the accepting section 16 (S26: YES), the threshold setting section 12 changes the thresholds for the passenger and/or adjusts the intensity and application period of a stimulus according to content of the passenger's report (S27).

For example, in a case where the passenger feels that he/she requires a stimulus while no stimulus is applied by the stimulus application device 6, the passenger uses the report device 7 to transmit, to the accepting section 16, a report stating that he/she requires application of a stimulus. Then, the threshold setting section 12 lowers the thresholds, thereby facilitating the stimulus application device 6 to apply a stimulus.

Meanwhile, in a case where the passenger feels that he/she does not require a stimulus while no stimulus is applied by the stimulus application device 6, the passenger uses the report device 7 to transmit, to the accepting section 16, a report stating that he/she does not require application of a stimulus. Further, in a case where the content of the report given by the passenger is not changed even after lapse of a traveling period of the vehicle 100, the threshold setting section 12 raises the thresholds, thereby making it difficult for the stimulus application device 6 to apply a stimulus.

In a case where the passenger feels that a stimulus is annoying while a stimulus is applied by the stimulus application device 6, the passenger uses the report device 7 to transmit, to the accepting section 16, a report stating that he/she does not require application of a stimulus. Then, the threshold setting section 12 raises the thresholds, thereby making it difficult for the stimulus application device 6 to apply a stimulus.

Meanwhile, in a case where the passenger feels that application of a stimulus is appropriate while a stimulus is applied by the stimulus application device 6, the passenger uses the report device 7 to transmit, to the accepting section 16, a report stating that he/she requires application of a stimulus. Further, in a case where the content of the report given by the passenger is not changed even after lapse of the traveling period of the vehicle 100, the threshold setting section 12 lowers the thresholds, thereby facilitating the stimulus application device 6 to apply a stimulus.

The passenger may manipulate the report device 7 so as to adjust an intensity and an application period of a stimulus applied by the stimulus application device 6. For example, the passenger may adjust an intensity of an air blow from the air conditioner and/or a vibration period of the vibrator.

Further, the passenger may manipulate the report device 7 to select any one of the “prevention mode”, the “ordinary mode”, and the “concentration mode”. For example, the passenger selects the “prevention mode” when he/she does not want to get motion sickness, and the passenger selects the “concentration mode” when he/she wants to concentrate on a task.

Meanwhile, if a report given by the passenger is not accepted by the accepting section 16 (S26: NO) or after step S27, steps S21 to S23 are repeated at given time intervals.

Subsequently, in step S28, the determination section 14 determines, on the basis of the information obtained from the car navigation system 4, whether or not the vehicle 100 has arrived at the destination. If the vehicle 100 has not arrived at the destination (S28: NO), steps S21 to S23 are carried out again.

Meanwhile, if the vehicle 100 has arrived at the destination (S28: YES), the motion sickness prevention process (S8) shown in FIG. 10 is ended.

Although not illustrated in the flowchart shown in FIG. 10, while the vehicle 100 is traveling, the pieces of biological information and the pieces of information related to the motion sickness characteristics and/or the like of the passengers are obtained in association with the pieces of ID information of the passengers, and are stored in the storage device 3.

With the motion sickness prevention device 1 in accordance with the first embodiment, it is assumed that a stimulus is applied to a passenger(s) by the stimulus application devices 6 more than once in one travel from the departure point to the destination; therefore, it can be expected to obtain, in one travel, many pieces of the above-described information.

Then, referring back to the flowchart in FIG. 3, after step S8, if the vehicle 100 arrives at the destination and the vehicle 100 stops traveling (S9) and then the driver stops the engine of the vehicle 100 (S10), the motion sickness prevention device 1 stops. Then, the control process shown in FIG. 2 is ended.

With the above-described motion sickness prevention device 1 in accordance with the first embodiment, if the determination section 14 determines that index values for a certain passenger are equal to or more than respective thresholds for the passenger (S24: YES), the control section 15 controls the stimulus application device 6 to apply a certain stimulus to the certain passenger (S25). This makes it possible to reduce a phenomenon that a factor which may cause motion sickness is accumulated in the passenger before the passenger himself/herself is not aware of it, thereby making it possible to prevent the passenger from getting motion sickness. This allows the passenger to keep riding on the vehicle without getting motion sickness. Consequently, the passenger can keep on a task of, e.g., manipulating a smartphone or playing a video game while staying concentrated on the task.

Further, the threshold setting section 12 sets the thresholds on the basis of the type of the vehicle 100 and the seating position of the passenger (S5). Thus, it is possible to apply a stimulus to the passenger at an appropriate timing, in consideration of differences in likelihood of getting motion sickness depending on the type of the vehicle 100 and the seating position of the seat. This makes it possible to reduce the possibility that each passenger may get motion sickness.

Further, when riding of a passenger on the vehicle is for the first time, the threshold setting section 12 sets thresholds on the basis of the biological information of the passenger obtained by the obtaining section 11 (S5). Meanwhile, when riding of a passenger on the vehicle is the second time or subsequent time, the threshold setting section 12 changes thresholds on the basis of the motion sickness characteristics of the passenger stored in the storage device 3 (S6). With this, it is possible to apply a stimulus to the passengers individually in a more appropriate manner, in consideration of the pieces of biological information and the motion sickness characteristics of the passengers. In a case where riding of a passenger on the vehicle is for the first time, the threshold setting section 12 may set thresholds for the passenger with respect to the respective behaviors of the vehicle 100 on the basis of a result of a questionnaire as to motion sickness which questionnaire is collected from the passenger in advance.

Moreover, the threshold setting section 12 changes the thresholds on the basis of the road information obtained by the obtaining section 11. For example, in a case where the vehicle travels on a road having many curves and a large degree of undulation or a road with traffic congestion, the threshold setting section 12 lowers the thresholds. This facilitates the stimulus application device 6 to apply a stimulus, thereby reliably preventing the passenger from getting motion sickness.

If the accepting section 16 accepts a report given by a passenger (S26: YES), the threshold setting section 12 changes the thresholds according to content of the report and/or adjusts an intensity and an application period of the stimulus (S27). With this, it is possible to adjust, according to passenger's wish, stimulus application carried out by the stimulus application device 6.

### Second Embodiment

Next, the following description will discuss, with reference to FIG. 12, a motion sickness prevention device 1 in accordance with a second embodiment of the present disclosure. For convenience of description, a member having a function identical to that of a member discussed in the first embodiment is given an identical reference sign, and a description thereof is omitted.

In the motion sickness prevention device 1 in accordance with the second embodiment, in a case where a degree of acceleration of a vehicle 100 obtained by an obtaining section 11 is equal to or more than a given value, a control section 15 controls a corresponding one(s) of stimulus application devices 6 to apply a stimulus to a passenger(s).

The “given value” is set at a value which is 0.8 times larger than the “threshold” set with respect to the respective kinds of behaviors of the vehicle 100 in the first embodiment, for example. The given value is set in this manner so as to consider, for example, the following circumstance. That is, while the vehicle 100 is traveling on a zigzag road, acceleration in a left-right direction is continuously applied to a passenger(s); therefore, the passenger(s) is/are more likely to get motion sickness, as compared to a case where the vehicle 100 travels on an ordinary road.

FIG. 12 is a view illustrating a timing that a stimulus is applied by the motion sickness prevention device 1 in accordance with the second embodiment. As shown in FIG. 12, in the second embodiment, the following situation is assumed. That is, while the vehicle 100 is traveling on a zigzag road, acceleration in the left-right direction is continuously applied to a passenger(s). In this case, due to repeated application of acceleration in the left-right direction to the passenger(s), a factor which may cause motion sickness is likely to be accumulated in the passenger(s).

In the second embodiment, in a case where a change in acceleration is detected a predetermined number or more of times, the control section 15 carries out control in the following manner. That is, at each of timings (X1 to X4 in FIG. 12) that a value of acceleration becomes equal to or more than 0.8 times larger than the threshold, the control section 15 controls a corresponding one(s) of the stimulus application device 6 to apply a given stimulus to the passenger(s).

As discussed above, the motion sickness prevention device 1 in accordance with the second embodiment is configured such that the control section 15 controls, according to a change in acceleration of the vehicle 100 obtained by the obtaining section 11, the stimulus application device(s) 6 to apply a stimulus to the passenger(s). With this, even in a case where a weak motion stimulus i repeatedly applied to the passengers, it is possible to reliably prevent the passengers from getting motion sickness.

Note that the calculation section 13 may calculate a cumulative value of acceleration applied to the vehicle 100 and the determination section 14 may determine whether or not the cumulative value of acceleration becomes equal to or more than a predetermined value. In this case, at a timing that the cumulative value of acceleration becomes equal to or more than the predetermined value, the control section 15 controls the stimulus application device(s) 6 to apply a given stimulus to the passenger(s). With this configuration, in a case where a weak shock is repeatedly applied to the passengers, it is possible to reliably prevent the passengers from getting motion sickness.

### Other Embodiments

In the above explanation, the motion sickness prevention devices 1 in accordance with the first and second embodiments are each configured such that the calculation section 13 calculates the index values with respect to the respective kinds of behaviors of the vehicle 100. However, this is not limitative. Alternatively, for example, the calculation section 13 may calculate a total index value which is a sum of the index values calculated with respect to the respective kinds of behaviors of the vehicle 100. In this cases, the threshold setting section 12 sets a total threshold corresponding to the total index value. The determination section 14 determines whether or not the total index value is equal to or more than the total threshold. Then, in a case where the determination section 14 determines that the total index value is equal to or more than the total threshold, the control section 15 controls the stimulus application device(s) 6 to apply a stimulus to the passenger(s).

With the above configuration, the stimulus application devices 6 are controlled in consideration of the total index value which is the sum of the index values calculated with respect to the respective kinds of behaviors of the vehicle 100. With this, a frequency of stimulus application by the stimulus application device 6 is optimized for each passenger. Thus, it is possible to prevent the passenger from getting motion sickness, while suppressing or reducing a situation in which the passenger is annoyed by the stimulus.

In the above explanation, the motion sickness prevention devices 1 in accordance with the first and second embodiments are each configured such that the control section 15 causes stimulus application device(s) 6 to apply a stimulus to the passenger(s) according to the information related to behaviors of the vehicle 100. In addition to this, in consideration of the feeling(s) of the passenger(s), the stimulus application device(s) 6 may be caused to carry out stimulus application.

In this case, for example, the calculation section 13 assumes the passengers' feelings on the basis of heart rates and respiration rates of the passengers detected by the vital sensor 25. Then, the determination section 14 determines whether or not each of the passengers' feelings assumed by the calculation section 13 is “uncomfortable”. If the determination section 14 determines that any one of the passengers' feeling is “uncomfortable”, the control section 15 controls a corresponding one(s) of the stimulus application devices 6 to apply a stimulus to the passenger(s). With this, it is possible to out carry appropriate stimulus application in consideration of the passengers' feelings.

Further, the calculation section 13 may assume whether the passengers' feelings are “comfortable” or “uncomfortable”. If it is determined that any of the passengers' feelings is/are “uncomfortable”, the thresholds may be raised by the threshold setting section 12. Meanwhile, if it is determined that any of the passengers' feelings is/are “comfortable”, the thresholds may not be changed.

In the above explanation, the motion sickness prevention devices 1 in accordance with the first and second embodiments are each configured such that the control section 15 causes the stimulus application device(s) 6 to give a stimulus to the passenger(s) before the passenger(s) get motion sickness. However, this is not limitative. For example, for a passenger who gets motion sickness, the control section 15 may increase an intensity of a stimulus and apply the stimulus for a longer period of time, so as to relieve the motion sickness of the passenger.

In the above explanation, the motion sickness prevention devices 1 in accordance with the first and second embodiments are each configured such that, if the vehicle 100 arrives at the destination (S28: YES), the motion sickness prevention process (S8) shown in FIG. 10 is ended. However, this is not limitative. For example, when the motion sickness prevention device 1 is turned off by passenger manipulation, the motion sickness prevention process (S8) may be ended.

### Software Implementation Example

The functions of the motion sickness prevention device 1 (hereinafter, referred to as a “device”) can be realized by a program for causing a computer to function as the device, the program causing the computer to function as the control blocks (the obtaining section 11, the threshold setting section 12, the calculation section 13, the determination section 14, the control section 15, and the accepting section 16) of the device.

In this case, the device includes, as hardware for executing the program, a computer that includes at least one control device (e.g., a processor) and at least one storage device (e.g., a memory). By the program with the control device and the storage device, the functions described in the foregoing embodiments are realized.

The program may be stored in one or more non-transitory computer-readable storage media. The one or more storage media may or may not be included in the device. In the latter case, the program may be supplied to or made available to the device via any wired or wireless transmission medium.

Alternatively, a part of or all of the functions of the control blocks can be realized by a logic circuit. For example, the present invention encompasses, in its scope, an integrated circuit in which a logic circuit that functions as each of the control blocks is formed. Alternatively, the functions of the control blocks can be realized, for example, by a quantum computer.

Each of the processes described in the foregoing embodiments may be carried out by artificial intelligence (AI). In this case, the AI may operate in the control device or may operate in another device (e.g., an edge computer or a cloud server).

The present disclosure is not limited to the embodiments, but can be altered by a skilled person in the art within the scope of the claims. The present invention also encompasses, in its technical scope, any embodiment derived by combining technical means disclosed in differing embodiments.

### REFERENCE SIGNS LIST

1: motion sickness prevention device
2: sensor group
3: storage device
4: car navigation system
5: communication device
6: stimulus application device
7: report device
8: external server
10: control device
11: obtaining section
12: threshold setting section
13: calculation section
14: determination section
15: control section
16: accepting section
21: camera
22: acceleration sensor
23: angular velocity sensor
24: velocity sensor
25: vital sensor
31: vehicle information
32: road information
33: passenger information
34: threshold information
100: vehicle

1. A motion sickness prevention device comprising:
a sensor group which is provided to a moving body and which detects information related to behaviors of the moving body;
an obtaining section which obtains the information related to the behaviors of the moving body detected by the sensor group;
a stimulus application device which applies a stimulus to a passenger of the moving body; and
a control section which controls the stimulus application device,
the control section controlling, according to the information related to the behaviors of the moving body obtained by the obtaining section, the stimulus application device so as to apply the stimulus to the passenger.

2. The motion sickness prevention device according to claim 1, further comprising:
a calculation section which calculates, on a basis of the information related to the behaviors of the moving body obtained by the obtaining section, index values each indicative of a likelihood of getting motion sickness; and
a determination section which determines whether or not the index values calculated by the calculation section are equal to or more than respective thresholds, wherein:
in a case where the determination section determines that at least one of the index values is equal to or more than a corresponding one of the thresholds, the control section controls the stimulus application device so as to apply the stimulus to the passenger.

3. The motion sickness prevention device according to claim 2, further comprising:
a threshold setting section which sets the threshold, wherein:
the calculation section calculates the index values with respect to respective kinds of the behaviors of the moving body;
the threshold setting section sets the thresholds with respect to the respective kinds of the behaviors of the moving body;
the determination section determines whether or not each of the index values calculated by the calculation section is equal to or more than one of the thresholds corresponding to the each of the index values; and
in a case where the determination section determines that any one of the index values is equal to or more than one of the thresholds corresponding to the one of the index values, the control section controls the stimulus application device so as to apply the stimulus to the passenger.

4. The motion sickness prevention device according to claim 3, wherein:
the calculation section calculates a total index value which is a sum of the index values calculated with respect to the respective kinds of the behaviors;
the threshold setting section sets a total threshold corresponding to the total index value;
the determination section determines whether or not the total index value calculated by the calculation section is equal to or more than the total threshold; and
in a case where the determination section determines that the total index value is equal to or more than the total threshold, the control section controls the stimulus application device so as to apply the stimulus to the passenger.

5. The motion sickness prevention device according to claim 3, wherein:
the thresholds are set on a basis of a type of the moving body and a seating position of the passenger.

6. The motion sickness prevention device according to claim 3, wherein:
the obtaining section obtains biological information of the passenger from the sensor group or an external server; and
the threshold setting section changes, on a basis of the biological information obtained by the obtaining section, the thresholds set by the threshold setting section.

7. The motion sickness prevention device according to claim 3, further comprising:
a storage device in which motion sickness characteristics of the passenger is stored, wherein:
the threshold setting section changes, on a basis of the motion sickness characteristics of the passenger stored in the storage device, the thresholds set by the threshold setting section.

8. The motion sickness prevention device according to claim 3, further comprising:
a navigation device provided to the moving body, wherein:
the obtaining section obtains, from the sensor group or the navigation device, road information indicative of a road condition of a road on which the moving body travels; and
the threshold setting section sets the thresholds on a basis of the road information obtained by the obtaining section.

9. The motion sickness prevention device according to claim 3, further comprising:
an accepting section which accepts a report, given by the passenger, related to control of the stimulus application device, wherein:
in a case where the accepting section accepts the report given by the passenger, the threshold setting section changes, according to content of the report, the thresholds set by the threshold setting section.

10. The motion sickness prevention device according to claim 1, wherein:
the sensor group includes at least one selected from the group consisting of an acceleration sensor which detects acceleration of the moving body, an angular velocity sensor which detects an angular velocity of the moving body, and a vital sensor which detects biological information of the passenger.

11. The motion sickness prevention device according to claim 2, wherein:
the sensor group includes an orientation sensor which detects an orientation of the passenger's body relative to the moving body;
the calculation section corrects the index values in consideration of the orientation of the passenger's body detected by the orientation sensor; and
the determination section determines whether or not each of the index values corrected by the calculation section is equal to or more than a corresponding one of the thresholds which are in accordance with the orientation of the passenger's body.

12. A motion sickness prevention method comprising:
detecting, by a sensor group provided to a moving body, information related to behaviors of the moving body;
obtaining the information related to the behaviors of the moving body detected in the detecting; and
applying a stimulus to a passenger of the moving body according to the information related to the behaviors of the moving body, the information being obtained in the obtaining.
